(19) United States
(12) Patent Application Publication
(10) Pub. No.: US 2025/0116765 A1
(43) Pub. Date: Apr. 10, 2025

(54) ULTRASOUND DIAGNOSTIC APPARATUS AND PROGRAM

(71) Applicant: FUJIFILM Corporation, Tokyo (JP)

(72) Inventors: Kenta INOUE, Tokyo (JP); Teiichiro IKEDA, Tokyo (JP); Takahide TERADA, Tokyo (JP)

(73) Assignee: FUJIFILM Corporation, Tokyo (JP)

(21) Appl. No.: 18/902,781

(22) Filed: Sep. 30, 2024

(30) Foreign Application Priority Data
Oct. 5, 2023 (JP) 2023-173449

(51) Int. Cl. G01S 7/52; A61B 8/00; G01S 15/89

(52) U.S. Cl. CPC G01S 7/52057; A61B 8/463; A61B 8/54; G01S 15/8915

(57) ABSTRACT

A recognition unit executes recognition processing of a scanning cross section on an ultrasound image generated by being scanned with ultrasound, to estimate a plurality of candidates for a scanning cross section scanned with the ultrasound. The recognition unit calculates a score indicating a certainty of the estimation for each candidate. A display controller displays, on a display, candidate images showing a plurality of respective candidates having higher scores among the plurality of candidates in different display aspects for each score.

### CROSS-REFERENCE TO RELATED APPLICATION

This application claims priority under 35 USC 119 from Japanese Patent Application No. 2023-173449, filed 5 Oct. 2023, the disclosure of which is incorporated by reference herein.

### BACKGROUND OF THE INVENTION

### 1. Field of the Invention

The present disclosure relates to an ultrasound diagnostic apparatus and a program.

### 2. Description of the Related Art

A technique for executing cross section recognition processing on an ultrasound image to estimate candidates for a cross section scanned with ultrasound and to display candidates having a likelihood equal to or higher than a threshold value is known.

JP2021-153774A discloses a technique for displaying candidates for a body mark and likelihoods thereof based on cross section information for the number of candidates.

### SUMMARY OF THE INVENTION

Meanwhile, in a case in which the displayed cross section recognition result is a result that is not intended by a user, the user may want to correct the cross section recognition result using an intuitive method. As a premise, the user may want to intuitively understand the cross section recognition result. However, in the related art, a method to satisfy such a request of the user cannot be provided.

An object of the present disclosure is to provide a technique for allowing a user to intuitively understand a cross section recognition result in a case in which cross section recognition processing is executed on an ultrasound image.

An aspect of the present disclosure provides an ultrasound diagnostic apparatus comprising: a recognition unit that executes recognition processing of a scanning cross section on an ultrasound image generated by being scanned with ultrasound, to estimate a plurality of candidates for a scanning cross section scanned with the ultrasound, and calculates a score indicating a certainty of the estimation for each candidate; and a display controller that displays, on a display, candidate images showing a plurality of respective candidates having higher scores among the plurality of candidates in different display aspects for each score.

The display controller may display a plurality of the candidate images on the display in an arranged manner in an arrangement order corresponding to the scores, and changes the arrangement order of the plurality of candidate images in response to an instruction issued by a user to change the arrangement order to display the plurality of candidate images in an arranged manner on the display.

The ultrasound diagnostic apparatus may further comprise: a reception unit that receives user's selection of a candidate having a score equal to or higher than a threshold value, and does not receive user's selection of a candidate having a score lower than the threshold value.

In a case in which a candidate that corresponds to a candidate image displayed at a position corresponding to a specific rank in the arrangement order corresponding to the scores and that has the score equal to or higher than the threshold value is selected by the user, the reception unit may receive the selection.

In a case in which the candidate having the score lower than the threshold value is selected by the user, the reception unit may receive the selection as valid selection depending on a content of processing executed after the selection.

In a case in which a plurality of ultrasound images showing candidates having scores equal to or higher than a first threshold value are consecutively acquired, and the number of the consecutively acquired plurality of ultrasound images is equal to or higher than a second threshold value, the ultrasound diagnostic apparatus may execute a next examination.

In a case in which a candidate having a score equal to or higher than a threshold value is not estimated, the display controller may display information prompting a user to manually select the candidate on the display.

In a case in which a candidate having a score equal to or higher than a threshold value is not estimated, and a region in which a plurality of the candidate images are displayed is designated, the display controller may display a candidate list for a user to select the candidate on the display.

The display controller may display a candidate for a cross section for which an examination is completed and a candidate for a cross section for which the examination is not completed, on the display in a distinguishable manner.

In a case in which a user issues an instruction to execute re-recognition after the plurality of candidates are estimated by the recognition unit, the recognition unit may execute the recognition processing on the ultrasound image again to estimate a new plurality of candidates for the scanning cross section, excluding a candidate having a highest score among the plurality of candidates. The display controller may display candidate images showing the new plurality of candidates on the display.

The ultrasound diagnostic apparatus may further comprise: an execution unit that executes an examination in accordance with a setting related to a first candidate among the plurality of candidates.

Another aspect of the present disclosure provides a program causing a computer to function as: a recognition unit that executes recognition processing of a scanning cross section on an ultrasound image generated by being scanned with ultrasound, to estimate a plurality of candidates for a scanning cross section scanned with the ultrasound, and calculates a score indicating a certainty of the estimation for each candidate; and a display controller that displays, on a display, candidate images showing a plurality of respective candidates having higher scores among the plurality of candidates in different display aspects for each score.

According to the present disclosure, it is possible to provide the technique for allowing the user to intuitively understand the cross section recognition result in a case in which the cross section recognition processing is executed on the ultrasound image.

### BRIEF DESCRIPTION OF THE DRAWINGS

FIG. 1 is a block diagram showing a configuration of an ultrasound diagnostic apparatus according to an embodiment.

FIG. 2 is a diagram showing a candidate image.

FIG. 3 is a diagram schematically showing a rotary encoder.

FIG. 4 is a diagram showing a display example of a candidate image group.

FIG. 5 is a diagram showing a display example of the candidate image group.

FIG. 6 is a diagram showing a selection screen displaying a candidate list for a scanning cross section.

FIG. 7 is a diagram showing a display example of the candidate image group.

FIG. 8 is a diagram showing a selection screen displaying the candidate list for the scanning cross section.

FIG. 9 is a diagram showing a display example of the candidate image group.

FIG. 10 is a block diagram showing a configuration of an ultrasound diagnostic apparatus according to Modification Example 5.

FIG. 11 is a diagram showing a display example of the candidate image group.

FIG. 12 is a diagram showing a determination screen for a user to determine a candidate.

FIG. 13 is a diagram showing a display example of a candidate image group.

FIG. 14 is a block diagram showing a configuration of an ultrasound diagnostic apparatus according to Modification Example 6.

### DESCRIPTION OF THE PREFERRED EMBODIMENTS

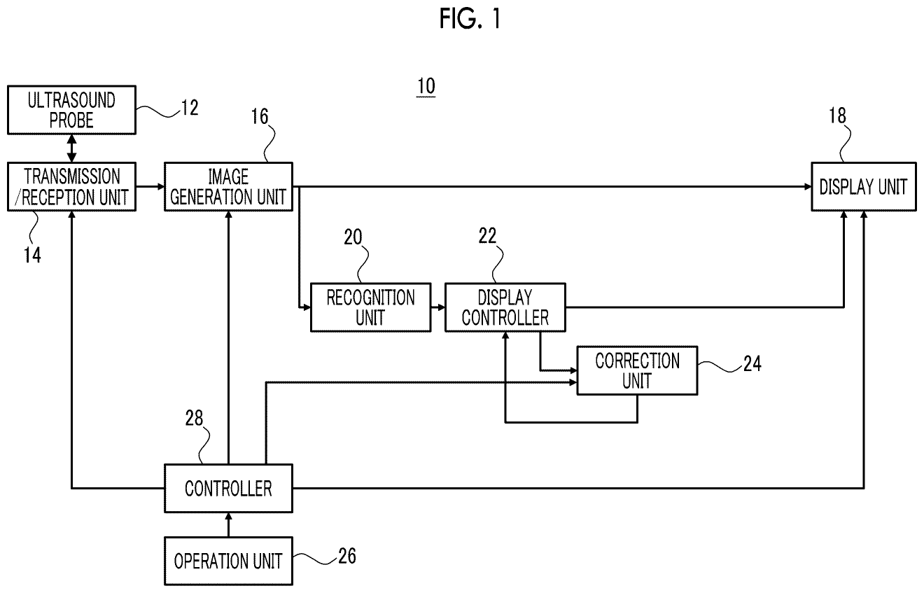

An ultrasound diagnostic apparatus 10 according to an embodiment will be described with reference to FIG. 1. FIG. 1 is a block diagram showing a configuration of the ultrasound diagnostic apparatus 10 according to the embodiment.

The ultrasound diagnostic apparatus 10 generates ultrasound image data by transmitting and receiving ultrasound using an ultrasound probe 12. For example, the ultrasound diagnostic apparatus 10 generates the ultrasound image data showing a tissue inside a subject by transmitting the ultrasound into the subject and receiving the ultrasound reflected from inside the subject.

The ultrasound probe 12 is a device that transmits and receives the ultrasound. The ultrasound probe 12 includes, for example, a 1D array transducer. The 1D array transducer is formed by arranging a plurality of ultrasound transducers in a one-dimensional array. An ultrasound beam is formed by the 1D array transducer, and electronic scanning with the ultrasound beam is repeatedly performed. As a result, a scanning cross section is formed in a living body for each electronic scanning. The scanning cross section corresponds to a two-dimensional echo data acquisition space. The ultrasound probe 12 may include a 2D array transducer formed by arranging a plurality of ultrasound transducers in a two-dimensional array. In a case in which the ultrasound beam is formed by the 2D array transducer and electronic scanning with the ultrasound beam is repeatedly performed, the scanning cross section as the two-dimensional echo data acquisition space is formed for each electronic scanning. In a case in which two-dimensional scanning with the ultrasound beam is performed, a three-dimensional space as a three-dimensional echo data acquisition space is formed. As a scanning method, sector scanning, linear scanning, convex scanning, or the like is used.

A transmission/reception unit 14 functions as a transmission beam former and a reception beam former. In a case of the transmission, the transmission/reception unit 14 supplies a plurality of transmission signals with a certain delay relationship to the plurality of ultrasound transducers included in the ultrasound probe 12. As a result, a transmission beam of the ultrasound is formed. In a case of the reception, a reflected wave (RF signal) from the inside of the living body is received by the ultrasound probe 12, whereby a plurality of reception signals are output from the ultrasound probe 12 to the transmission/reception unit 14. The transmission/reception unit 14 forms a reception beam by applying phasing addition processing to the plurality of reception signals. Data of the reception beam is output to an image generation unit 16. That is, the transmission/reception unit 14 forms the reception beam by executing delay processing on the reception signal obtained from each ultrasound transducer in accordance with a delay processing condition for each ultrasound transducer, and executing addition processing on the plurality of reception signals obtained from the plurality of ultrasound transducers. The delay processing condition is defined by reception delay data indicating a delay time. A reception delay data set (that is, a set of delay times) corresponding to the plurality of ultrasound transducers is supplied from a controller 28.

The electronic scanning with the ultrasound beam (that is, the transmission beam and the reception beam) is performed by the operation of the transmission/reception unit 14, thereby forming the scanning cross section. The scanning cross section corresponds to a plurality of beams, and the plurality of beams constitute a reception frame (specifically, an RF signal frame). It should be noted that each beam is formed of a plurality of echoes arranged in a depth direction. A plurality of reception frames arranged on a time axis are output from the transmission/reception unit 14 to the image generation unit 16 by repeating the electronic scanning with the ultrasound beam. The plurality of reception frames constitute a reception frame sequence.

In a case in which the two-dimensional electronic scanning with the ultrasound beam is performed by the operation of the transmission/reception unit 14, the three-dimensional echo data acquisition space is formed, and volume data as an echo data set is acquired from the three-dimensional echo data acquisition space. A plurality of volume data arranged on a time axis are output from the transmission/reception unit 14 to the image generation unit 16 by repeating the electronic scanning with the ultrasound beam. The plurality of volume data constitute a volume data sequence.

The image generation unit 16 generates ultrasound image data (for example, B-mode image data) by applying signal processing such as detection, amplitude compression (for example, logarithmic compression), and conversion functions (coordinate conversion function and interpolation processing function via a digital scan converter (DSC)) to the reception frame output from the transmission/reception unit 14. Hereinafter, the image data will be referred to as “image” as appropriate. For example, the ultrasound image data will be referred to as “ultrasound image”, and the B-mode image data will be referred to as “B-mode image” as appropriate. It should be noted that the ultrasound image according to the present embodiment is not limited to the B-mode image, and may be any image generated by the ultrasound diagnostic apparatus 10. For example, the ultrasound image according to the present embodiment may be a color Doppler image, a pulse Doppler image, a strain image, or a shear wave elastography image. The image generation unit 16 may execute processing of overlaying required graphic data onto the ultrasound image.

The ultrasound image is output to a display unit 18 and displayed on the display unit 18 in a display aspect in accordance with a display mode. The display mode is determined in advance. The user may change the display mode.

The display unit 18 is a display such as a liquid crystal display or an EL display. The ultrasound image such as the B-mode image is displayed on the display unit 18. The display unit 18 may be a device comprising both a display and an operation unit 26. For example, a graphic user interface (GUI) may be realized by the display unit 18. In addition, a user interface such as a touch panel may be realized by the display unit 18.

A recognition unit 20 estimates a plurality of candidates for the scanning cross section scanned with the ultrasound by executing cross section recognition processing on the ultrasound image. In addition, the recognition unit 20 calculates a score (that is, a degree of accuracy of the estimation) indicating a certainty of the estimation for each candidate for the scanning cross section. The score indicating the certainty of the estimation may also be referred to as a likelihood.

The recognition unit 20 may estimate the candidates for the scanning cross section currently being scanned by executing the cross section recognition processing on the currently acquired ultrasound image. That is, the recognition unit 20 may estimate the candidates for the scanning cross section in real time.

As another example, the recognition unit 20 may estimate the candidate for the scanning cross section by executing the cross section recognition processing on the ultrasound image that is already acquired and stored in a storage device of the ultrasound diagnostic apparatus 10 or an external apparatus.

Estimating the candidates for the scanning cross section is to estimate candidates for a part shown by the ultrasound image of the scanning cross section. That is, the recognition unit 20 estimates the candidates for the part being scanned with the ultrasound by executing the cross section recognition processing on the ultrasound image.

As the cross section recognition processing according to the present embodiment, known cross section recognition processing is used. For example, machine learning or artificial intelligence (AI) may be used in the cross section recognition processing. A type of the machine learning or the artificial intelligence to be used is not limited, and any algorithm or model may be used. For example, a convolutional neural network (CNN), a recurrent neural network (RNN), generative adversarial networks (GAN), linear models, decision tree learning, a support vector machine (SVM), an ensemble classifier, or other algorithms may be used. In addition, pattern matching algorithms such as template matching or algorithms that do not require learning, such as correlation coefficient or similarity calculation, may be used in the cross section recognition processing.

For example, the recognition unit 20 executes the cross section recognition processing using the machine learning on the ultrasound image to estimate the plurality of candidates for the scanning cross section, and calculates the score (that is, a degree of accuracy of the estimation) indicating the certainty of the estimation using the machine learning for each candidate for the scanning cross section. As a result, the plurality of candidates for the part being scanned with the ultrasound are estimated, and the score indicating the certainty of the estimation is calculated for each candidate for the part.

The recognition unit 20 may estimate the plurality of candidates for the scanning cross section scanned with the ultrasound by comparing the ultrasound image (for example, the B-mode image) generated by transmitting and receiving the ultrasound with a plurality of reference cross section images (for example, the B-mode images), and calculate a score indicating the certainty of the estimation for each candidate for the scanning cross section. In this case, a technique such as the pattern matching may be used.

The reference cross section image is the ultrasound image used for estimating the scanning cross section. For example, one or a plurality of reference cross section images are generated in advance for each diagnostic part and stored in the storage device of the ultrasound diagnostic apparatus 10 or the external apparatus. The reference cross section image showing a certain part is an ultrasound image generated by scanning a reference scanning cross section that intersects the part with the ultrasound. For example, the reference scanning cross section is a cross section to be imaged in an ultrasound examination, a representative cross section, or the like.

The maximum number of the candidates for the scanning cross section may be determined. The maximum number of the candidates for the scanning cross section may be determined for each part. In a case in which “25” is determined as the maximum number of the candidates, the recognition unit 20 estimates at maximum 25 candidates.

A display controller 22 displays, on the display unit 18, candidate images showing a plurality of respective candidates having higher scores among the plurality of candidates in different display aspects for each score. For example, the display controller 22 displays, on the display unit 18, a plurality of candidate images in an arranged manner in an arrangement order corresponding to the scores.

A correction unit 24 changes the arrangement order of the plurality of candidate images in response to a user's instruction to change the arrangement order. The correction unit 24 outputs information indicating the changed arrangement order to the display controller 22. For example, in a case in which the user issues the instruction to change the arrangement order to the ultrasound diagnostic apparatus 10 by operating the operation unit 26, the correction unit 24 changes the arrangement order of the plurality of candidate images in response to the change instruction.

In a case in which the information indicating the changed arrangement order is received from the correction unit 24, the display controller 22 displays the plurality of candidate images in an arranged manner in the changed arrangement order on the display unit 18. That is, the display controller 22 changes the arrangement order of the plurality of candidate images in response to the user's instruction to change the arrangement order, and displays the plurality of candidate images in an arranged manner on the display unit 18.

In a case in which a plurality of candidates having the scores equal to or higher than a threshold value are estimated, the display controller 22 may display the candidate for the scanning cross section for which the examination is completed and the candidate for the scanning cross section for which the examination is not completed, on the display unit 18 in a distinguishable manner. For example, the threshold value is determined in advance. The user may change the threshold value.

The operation unit 26 is a device for the user to input conditions, instructions, and the like required for the imaging or the examination into the ultrasound diagnostic apparatus 10. For example, the operation unit 26 may be an operation panel, a switch, a button, a keyboard, a mouse, a trackball, or a joystick.

The controller 28 controls the operations of the respective units of the ultrasound diagnostic apparatus 10. The controller 28 may receive the user's selection of the candidate having the score equal to or higher than the threshold value, and not receive the user's selection of the candidate having the score lower than the threshold value. In this case, the controller 28 functions as an example of a reception unit.

In a case in which a candidate that corresponds to a candidate image displayed at a position corresponding to a specific rank in the arrangement order corresponding to the scores and that has the score equal to or higher than the threshold value is selected by the user, the controller 28 may receive the selection.

For example, the user selects the candidates for the scanning cross section by operating the operation unit 26. The controller 28 receives the selection or does not receive the selection.

The ultrasound diagnostic apparatus 10 includes the storage device (not shown). The storage device is a device that forms one or a plurality of storage regions for storing data. The storage device is, for example, a hard disk drive (HDD), a solid-state drive (SSD), various memories (for example, a RAM, a DRAM, and a ROM), other storage devices (for example, an optical disk), or a combination thereof. For example, the information indicating the ultrasound image or the imaging condition is stored in the storage device.

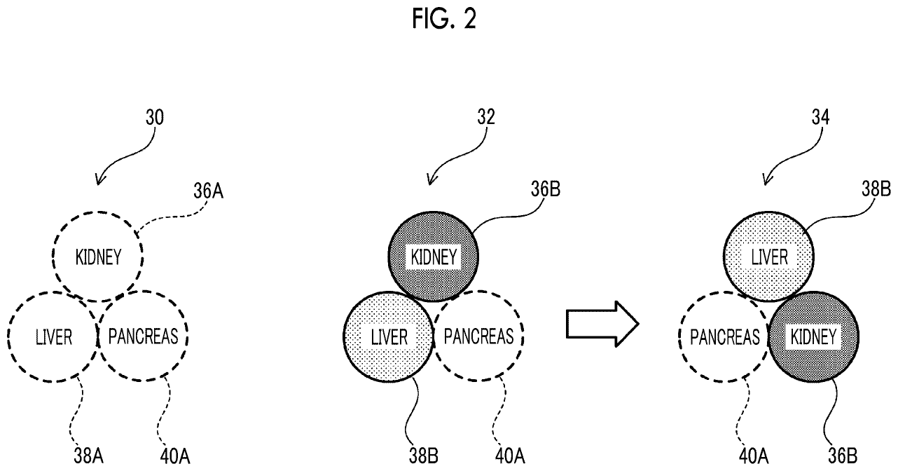

Hereinafter, the candidate image will be described with reference to FIG. 2. FIG. 2 shows an example of the candidate image.

Here, as an example, the recognition unit 20 estimates a kidney cross section, a liver cross section, and a pancreas cross section as the candidates for the scanning cross section. The kidney cross section image is a scanning cross section that intersects a kidney. The liver cross section is a scanning cross section that intersects a liver. The pancreas cross section image is a scanning cross section that intersects a pancreas. That is, the recognition unit 20 estimates that any one of the kidney, the liver, or the pancreas is shown in the ultrasound image generated by transmitting and receiving the ultrasound. In other words, the recognition unit 20 estimates that any one of the kidney, the liver, or the pancreas is being scanned with the ultrasound.

In addition, the recognition unit 20 calculates the score indicating the certainty of the estimation for each candidate for the scanning cross section. Here, the recognition unit 20 calculates the score indicating the certainty of the estimation of the kidney cross section, the score indicating the certainty of the estimation of the liver cross section, and the score indicating the certainty of the estimation of the pancreas cross section.

The display controller 22 displays, on the display unit 18, the candidate images showing the plurality of respective candidates having higher scores among the plurality of candidates in different display aspects for each score. For example, the display controller 22 displays, on the display unit 18, the plurality of candidate images in an arranged manner in the arrangement order corresponding to the scores. For example, the candidates ranked from first to third places are higher-rank candidates. Here, as an example, the kidney cross section, the liver cross section, and the pancreas cross section are candidates having higher scores.

The recognition unit 20 may recognize other scanning cross sections other than the kidney cross section, the liver cross section, and the pancreas cross section, and may calculate the scores for these other scanning cross sections. In this case as well, the higher-rank candidate images are displayed on the display unit 18. For example, in a case in which the kidney cross section, the liver cross section, and the pancreas cross section are the higher-rank candidates among the kidney cross section, the liver cross section, the pancreas cross section, and the other scanning cross sections, the candidate images of each of the kidney cross section, the liver cross section, and the pancreas cross section are displayed on the display unit 18.

FIG. 2 shows candidate image groups 30, 32, and 34. Each candidate image group includes the plurality of candidate images. Hereinafter, each of the candidate image groups 30, 32, and 34 will be described.

First, the candidate image group 30 will be described. The candidate image group 30 includes candidate images 36A, 38A, and 40A. The candidate image 36A is an image showing the kidney cross section that is the candidate. The candidate image 38A is an image showing the liver cross section that is the candidate. The candidate image 40A is an image showing the pancreas cross section that is the candidate.

The display controller 22 disposes each candidate image at a position corresponding to the score in the candidate image group 30. For example, among the kidney cross section, the liver cross section, and the pancreas cross section, the score of the kidney cross section is the highest, the score of the liver cross section is the second highest, and the score of the pancreas cross section is the third highest. The scanning cross section having the highest score is a first candidate, the scanning cross section having the second highest score is a second candidate, and the scanning cross section having the third highest score is a third candidate. The display controller 22 disposes the candidate image 36A, which is the first candidate, at a first candidate position corresponding to the highest score. The display controller 22 disposes the candidate image 38A, which is the second candidate, at a second candidate position corresponding to the second highest score. The display controller 22 disposes the candidate image 40A, which is the third candidate, at a third candidate position corresponding to the third highest score. In the example shown in FIG. 2, the first candidate position is a highest position among the three positions. The second candidate position is a position on a left side in the facing direction. The third candidate position is a position on a right side in the facing direction. Therefore, the display controller 22 disposes the candidate image 36A at the highest position, disposes the candidate image 38A at the position on the left side in the facing direction, and disposes the candidate image 40A at the position on the right side in the facing direction. As described above, the display controller 22 disposes the candidate images 36A, 38A, and 40A in an arranged manner in the arrangement order corresponding to the scores. By disposing and displaying each candidate image in this way, the user can intuitively understand the rank of each candidate for the scanning cross section.

The display controller 22 may distinguish between the display aspect of the candidate image showing the candidate having the score equal to or higher than the threshold value and the display aspect of the candidate image showing the candidate having the score lower than the threshold value. For example, the display controller 22 represents a frame of the candidate image showing the candidate having the score equal to or higher than the threshold value by a solid line, and represents a frame of the candidate image showing the candidate having score lower than the threshold value by a broken line. In this manner, the user can intuitively understand the candidate having the score equal to or higher than the threshold value and the candidate having the score lower than the threshold value in a distinguishable manner.

The frames of the candidate images 36A, 38A, and 40A included in the candidate image group 30 are represented by a broken line. That is, the scores of the kidney cross section, the liver cross section, and the pancreas cross section are lower than the threshold value. The fact that the score is lower than the threshold value means that a reliability degree of the estimation of each candidate is low. The user can recognize that the reliability degree of the estimation for each candidate is low by referring to the candidate image group 30. In addition, since the candidate having the score lower than the threshold value is also presented to the user, the user can understand what kind of scanning cross section the candidate having the score lower than the threshold value represents.

The display controller 22 displays the candidate image group 30 on the display unit 18. The user can intuitively understand the rank of the candidate for each scanning cross section (that is, the rank based on the score) by referring to the candidate image group 30. In addition, the user can intuitively understand that the reliability degree of the estimation of the candidate is low. In this case, it is considered that the user continues the imaging by changing a position or an angle of the ultrasound probe 12 such that the score corresponding to the part to be imaged (that is, the score of the scanning cross section that intersects the part to be imaged) is increased.

Hereinafter, the candidate image group 32 will be described. The candidate image group 32 includes candidate images 36B, 38B, and 40A. The candidate image 36B is an image showing the kidney cross section that is the candidate. The candidate image 38B is an image showing the liver cross section that is the candidate. The candidate image 40A is an image showing the pancreas cross section that is the candidate.

For example, among the kidney cross section, the liver cross section, and the pancreas cross section, the score of the kidney cross section is the highest, the score of the liver cross section is the second highest, and the score of the pancreas cross section is the third highest. Therefore, the display controller 22 disposes the candidate image 36B at the highest position, disposes the candidate image 38B at the position on the left side in the facing direction, and disposes the candidate image 40A at the position on the right side in the facing direction. As described above, the display controller 22 disposes the candidate images 36B, 38B, and 40A in an arranged manner in the arrangement order corresponding to the scores.

The frames of the candidate images 36B and 38B included in the candidate image group 32 are represented by a solid line. That is, the scores of the kidney cross section and the liver cross section are equal to or higher than the threshold value. The fact that the score is equal to or higher than the threshold value means that the reliability degree of the estimation of each candidate is high. On the other hand, as in the candidate image group 30, the frame of the candidate image 40A is represented by a broken line.

The display controller 22 displays the candidate image group 32 on the display unit 18. The user can intuitively understand that the scores of the kidney cross section and the liver cross section are equal to or higher than the threshold value, and the score of the pancreas cross section is lower than the threshold value by referring to the candidate image group 32.

For example, in a case in which the user operates the operation unit 26 to issue an instruction to execute a next examination, the controller 28 receives the instruction. The ultrasound diagnostic apparatus 10 executes the next examination in response to the instruction. For example, the ultrasound diagnostic apparatus 10 executes an examination specific to the part corresponding to the candidate image disposed at the first candidate position. Examples of the examination include measurement on the ultrasound image, setting of a Doppler flow velocity measurement, detection of an abnormality shown in the ultrasound image, display of the abnormality, adjustment of an image quality of the ultrasound image, creation of a report, and display of the report.

With reference to the candidate image group 32, the candidate image 36B corresponding to the kidney is disposed at the first candidate position. Therefore, in a case in which the user issues the instruction to execute the examination, the ultrasound diagnostic apparatus 10 executes the examination specific to the kidney on the ultrasound image. For example, a measurement specific to the kidney or the like is executed.

As another example, in a case in which the candidate that corresponds to the candidate image displayed at the position corresponding to the specific rank in the arrangement order corresponding to the scores and that has the score equal to or higher than the threshold value is selected by the user, the controller 28 may receive the selection. For example, the specific rank is a first rank (that is, a rank having the highest score), and the position corresponding to the specific rank is the first candidate position. In a case in which the user selects the candidate image 36B disposed at the first candidate position, the controller 28 receives the selection. In a case in which the user issues the instruction to execute the examination, the ultrasound diagnostic apparatus 10 executes the examination specific to the kidney corresponding to the candidate image 36B.

Even in a case in which the user selects the candidate image 38B disposed at the second candidate position, the controller 28 does not receive the selection. In the same manner, even in a case in which the user selects the candidate image 40A disposed at the third candidate position, the controller 28 does not receive the selection. Therefore, even in a case in which the user issues the instruction to execute the examination, the ultrasound diagnostic apparatus 10 does not execute the examination specific to the liver and the examination specific to the pancreas.

Further, as still another example, in a case in which the user selects the candidate image showing the candidate having the score equal to or higher than the threshold value by operating the operation unit 26, the controller 28 may receive the selection. In this case, the ultrasound diagnostic apparatus 10 executes the examination for the part corresponding to the selected candidate under the control of the controller 28. For example, in a case in which the user selects the candidate image 36B or the candidate image 38B by operating the operation unit 26, the controller 28 receives the selection. In a case in which the candidate image 36B is selected, the ultrasound diagnostic apparatus 10 executes the examination for the kidney. In a case in which the candidate image 38B is selected, the ultrasound diagnostic apparatus 10 executes the examination for the liver.

On the other hand, in a case in which the user selects the candidate image showing the candidate having the score lower than the threshold value by operating the operation unit 26, the controller 28 does not receive the selection. For example, even in a case in which the user selects the candidate image 40A, the controller 28 does not receive the selection. In this case, the ultrasound diagnostic apparatus 10 does not execute the examination for the pancreas.

Hereinafter, the candidate image group 34 will be described. The arrangement order of the candidate images 36B, 38B, and 40A included in the candidate image group 32 is changed, whereby the candidate image group 34 is generated.

In a case in which the user issues the instruction to change the arrangement order of the candidate images 36B, 38B, and 40A included in the candidate image group 32 by operating the operation unit 26, the correction unit 24 changes the arrangement order of the candidate images 36B, 38B, and 40A in response to the instruction. The display controller 22 displays the candidate image group 34 in a state in which the arrangement order of the candidate images is changed, on the display unit 18.

For example, by the user's operation, the candidate image 38B is disposed at the first candidate position, the candidate image 40A is disposed at the second candidate position, and the candidate image 36B is disposed at the third candidate position. In this state, in a case in which the user issues the instruction to execute the examination, the ultrasound diagnostic apparatus 10 executes the examination specific to the liver corresponding to the candidate image 38B disposed at the first candidate position.

In a case in which the user selects the candidate image showing the candidate having the score lower than the threshold value by operating the operation unit 26, the controller 28 may receive the selection as valid selection depending on a content of processing executed after the selection. For example, a workflow of the ultrasound examination is determined in advance. The workflow of the ultrasound examination is an examination protocol or an examination procedure. The workflow includes a plurality of examinations (for example, the measurement or the like). An order in which each examination is executed is determined in advance. Therefore, the examination executed after the selection of the candidate image is determined in advance. For example, the workflow is determined for each diagnostic target part, or the workflow is determined for each patient. In a case in which the examination executed after the selection of the candidate image is processing other than the measurement, the controller 28 may receive the selection as the valid selection. For example, processing of recording the position of the scanning cross section is an example of the examination other than measurement. In this case, the controller 28 receives the selection of the candidate image showing the candidate having the score lower than the threshold value as the valid selection, and records the position of the selected candidate (that is, the candidate for the scanning cross section). In a case in which the examination executed after the selection of the candidate image is the measurement, the controller 28 does not receive the selection as the valid selection. In this case, the measurement is not executed. It should be noted that, even in a case in which the examination executed after the selection of the candidate image is the measurement, the controller 28 may receive the selection as the valid selection. In this case, the ultrasound diagnostic apparatus 10 executes an examination specific to the part corresponding to the selected candidate. The display unit 18 displays the result of the examination. The display controller 22 displays a message indicating that the reliability degree is low on the display unit 18 together with the result of the examination.

For example, the user can change the arrangement order of the candidate images by operating a rotary encoder, a button or switch displayed on the touch panel, or a physical button or switch. The arrangement order of the candidate images may be changed by voice, eye gaze, or the like.

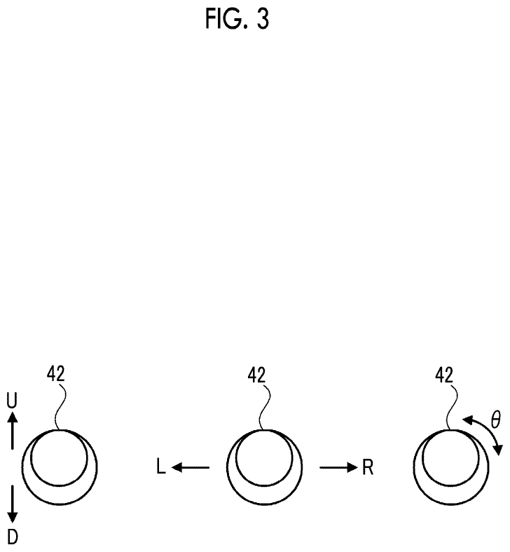

FIG. 3 shows a rotary encoder 42. The rotary encoder 42 is provided on an operation panel included in the operation unit 26. For example, the user can change the arrangement order of the candidate images by operating the rotary encoder 42 in an up direction (U direction), a down direction (D direction), a left direction (L direction), a right direction (R direction), and a rotation direction (0 direction).

For example, the candidate image can be moved in a direction in which the rotary encoder 42 is turned. Specifically, by turning the rotary encoder 42 clockwise, the candidate image disposed at the first candidate position can be disposed to the third candidate position, the candidate image disposed at the second candidate position can be disposed to the first candidate position, and the candidate image disposed at the third candidate position can be disposed to the second candidate position. In a case in which the user presses a confirm button after the arrangement order of the candidate images is changed, the change of the arrangement order may be confirmed.

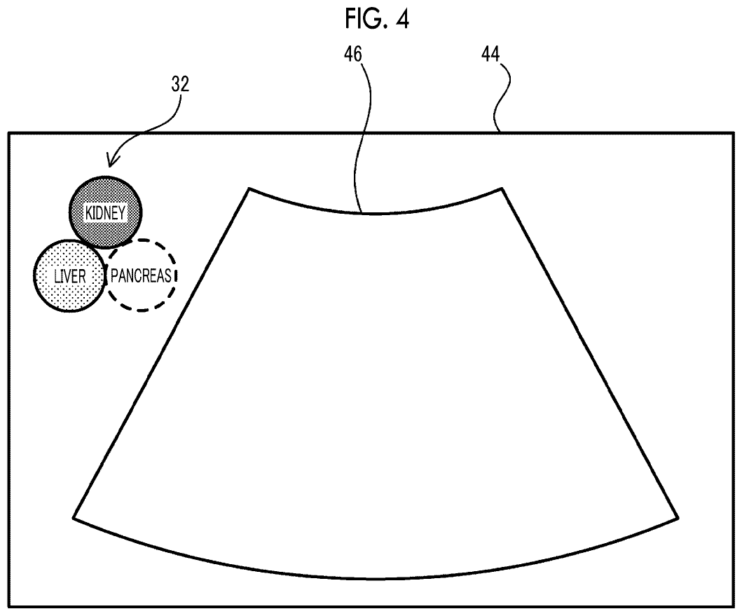

FIG. 4 shows a display example of the candidate image group. A screen 44 is formed on the display unit 18. For example, the display controller 22 displays an ultrasound image 46, such as the B-mode image, on the screen 44. Further, the display controller 22 displays the candidate image group of the scanning cross section (that is, the cross section from which the ultrasound image 46 is obtained) on the screen 44. Here, as an example, the screen 44 displays the candidate image group 32.

For example, the candidate image group is updated after the ultrasound image 46 is displayed in a freeze state. Displaying the ultrasound image 46 in a freeze state is to display the ultrasound image 46 as a still image. For example, in a case in which the user issues a freeze instruction by operating the operation unit 26, the display controller 22 displays the ultrasound image 46 in a freeze state on the display unit 18. A button or a switch for issuing the freeze instruction may be provided in the operation unit 26.

The recognition unit 20 estimates the plurality of candidates for the scanning cross section from which the ultrasound image 46 is obtained by executing the cross section recognition processing on the ultrasound image 46 in a freeze state, and calculates the score for each candidate. In the example shown in FIG. 4, the result of the estimation is displayed as the candidate image group 32.

The user can change the arrangement order of the candidate images included in the candidate image group 32 by operating the operation unit 26 while referring to the ultrasound image 46. For example, in a case in which the user issues the instruction to execute the examination in a state in which the candidate image 36B is disposed at the first candidate position, the ultrasound diagnostic apparatus 10 executes the examination specific to the kidney on the ultrasound image 46.

In this way, with the ultrasound diagnostic apparatus 10 according to the present embodiment, in a case in which the cross section recognition processing is executed on the ultrasound image, the user can intuitively understand the cross section recognition result by referring to the candidate image group. In addition, the user can intuitively change the cross section recognition result by changing the disposition position of each candidate image included in the candidate image group. As a result, the examination intended by the user can be executed on the ultrasound image. That is, the examination executed after the cross section recognition can be prevented from being the examination not intended by the user.

For example, in the diagnosis of renal cell carcinoma, the kidney cross section is extracted, and then a property in the renal cortex are diagnosed. In the present embodiment, after the plurality of candidates for the scanning cross section are estimated by the cross section recognition processing, the user can select the kidney cross section, which is an examination target, by changing the rank of each candidate. The kidney cross section is selected, and then the examination specific to the kidney is executed. For example, a region in the renal cortex is detected by a lesion region detection engine, a region suspected to be cell cancer in the renal cortex is detected by a property determination engine, and the property of the region is determined.

### Modification Example 1

Hereinafter, Modification Example 1 will be described. In Modification Example 1, in a case in which it is presumed that there is no need to change the arrangement order of the candidate images, even in a case in which the user does not issue the instruction to execute the examination, the ultrasound diagnostic apparatus 10 executes the examination specific to the part corresponding to the candidate image disposed at the first candidate position. With reference to the example shown in FIG. 4, even in a case in which the user does not issue the instruction to execute the examination, the ultrasound diagnostic apparatus 10 executes the examination specific to the kidney on the ultrasound image 46.

Specifically, in a case in which a plurality of ultrasound images showing the candidates having the scores equal to or higher than the first threshold value are consecutively acquired, and the number of the consecutively acquired plurality of ultrasound images is equal to or higher than a second threshold value, the controller 28 causes the ultrasound diagnostic apparatus 10 to execute the next examination. The ultrasound diagnostic apparatus 10 executes the examination specific to the part corresponding to the candidate image disposed at the first candidate position on the ultrasound image 46. The first threshold value and the second threshold value are values determined in advance. The user may change the first threshold value and the second threshold value.

More specifically, in a case (1) in which a change amount of the ultrasound image is equal to or lower than the threshold value over the number of frames equal to or higher than a predetermined number of frames, the controller 28 determines that the ultrasound probe 12 is stationary. That is, it is presumed that the ultrasound probe 12 is disposed at the same position, and the same cross section is being scanned with the ultrasound.

Next, in a case (2) in which the estimated candidates are the same over the number of frames equal to or higher than the predetermined number of frames, and the score of the first candidate is equal to or higher than the threshold value, the controller 28 determines that the estimation of the cross section is stable.

In a case in which the conditions (1) and (2) are satisfied, even in a case in which the user issuing the instruction to execute the examination, the ultrasound diagnostic apparatus 10 executes the examination specific to the part corresponding to the candidate image disposed at the first candidate position on the ultrasound image 46. That is, in a case in which it is determined that the ultrasound probe 12 is stationary and the estimation of the cross section is stable, even in a case in which the user issuing the instruction to execute the examination, the ultrasound diagnostic apparatus 10 executes the specific examination on the ultrasound image 46. It is possible to reduce the workload of the user.

### Modification Example 2

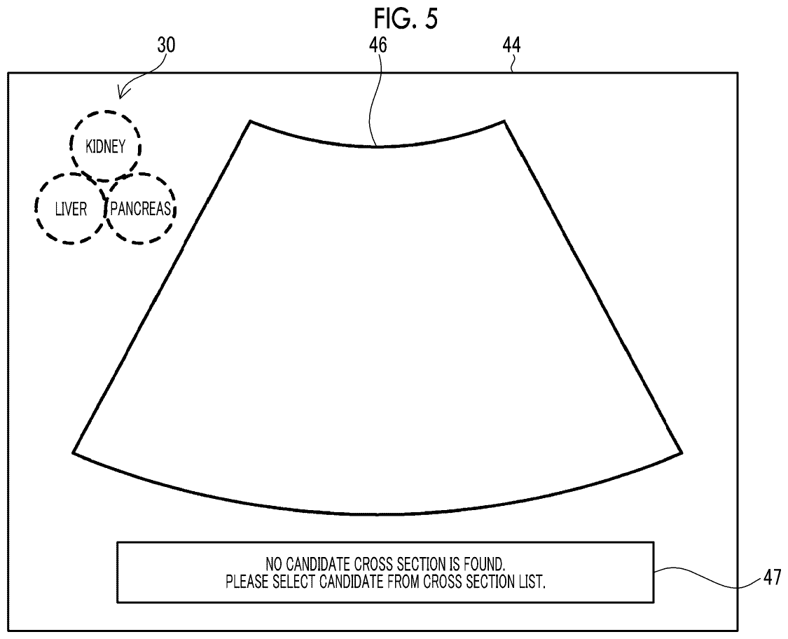

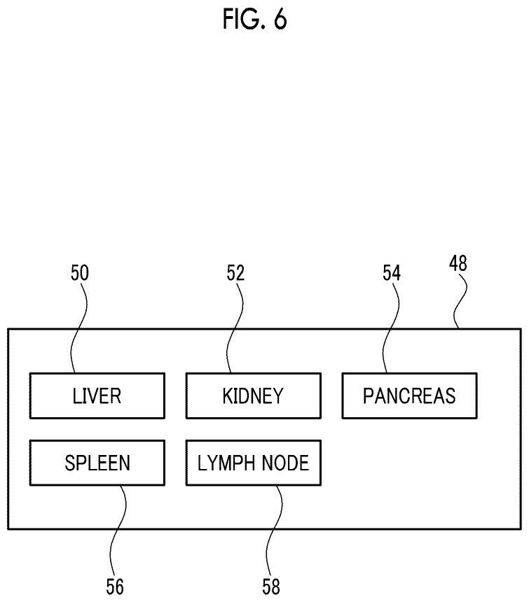

Hereinafter, Modification Example 2 will be described. In Modification Example 2, in a case in which the candidate having the score equal to or higher than the threshold value is not estimated, the display controller 22 displays the information prompting the user to manually select the candidates for the scanning cross section on the display unit 18. Modification Example 2 will be described in detail with reference to FIGS. 5 and 6. FIG. 5 shows a display example of the candidate image group. FIG. 6 shows a selection screen displaying a candidate list for the scanning cross section.

As shown in FIG. 5, the screen 44 displays the ultrasound image 46 and the candidate image group 30. Here, the scores of the kidney cross section, the liver cross section, and the pancreas cross section are lower than the threshold value. Therefore, the screen 44 displays the candidate image group 30.

In this case, the display controller 22 displays a message 47 prompting the user to manually select the candidate for the scanning cross section on the screen 44. For example, the message 47 such as “No candidate cross section is found. Please select a candidate from the cross section list.” is displayed on the screen 44.

In a case in which the user operates the operation unit 26 to issue an instruction to display the candidate list for the scanning cross section, the display controller 22 displays a selection screen 48 shown in FIG. 6 on the display unit 18. The selection screen 48 may be displayed on the display unit 18 in a case in which the user touches or clicks the message 47 on the screen 44.

The selection screen 48 displays scanning cross section names 50 to 58. Names of all candidates may be displayed on the selection screen 48, or the names of candidates selected through learning or the names of candidates based on a history may be displayed on the selection screen 48. The number of displayed names may be changed depending on a portion scanned with the ultrasound. Here, as an example, the liver, the kidney, the pancreas, a spleen, and a lymph node are displayed as the candidates for the scanning cross section.

In a case in which the user selects the scanning cross section name on the selection screen 48, the controller 28 confirms the scanning cross section having the selected scanning cross section name, as the cross section from which the ultrasound image 46 is obtained. For example, the ultrasound diagnostic apparatus 10 executes an examination specific to the scanning cross section having the selected scanning cross section name on the ultrasound image 46. The ultrasound diagnostic apparatus 10 may execute the examination even in a case in which the user does not issue the instruction to execute the examination. It goes without saying that the ultrasound diagnostic apparatus 10 may execute the examination in a case in which the user issues the instruction to execute the examination.

### Modification Example 3

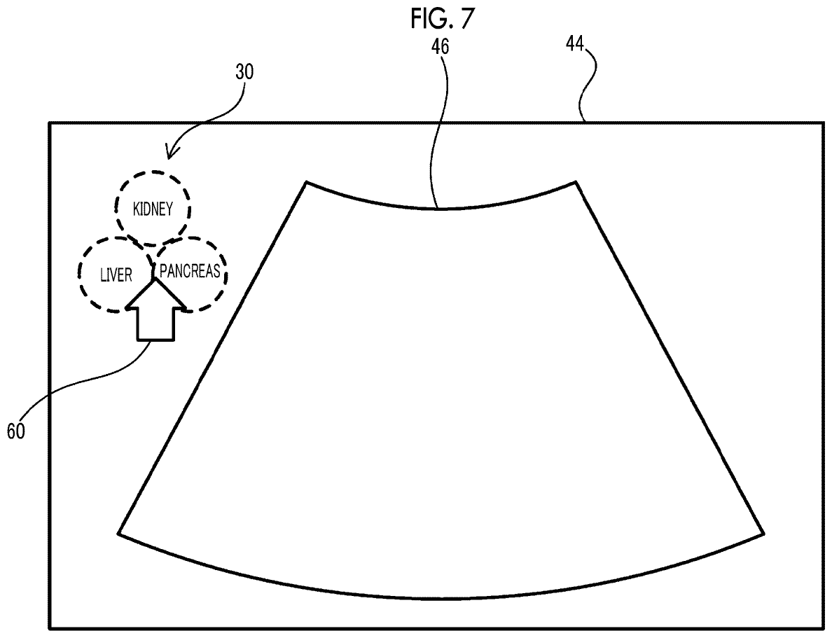

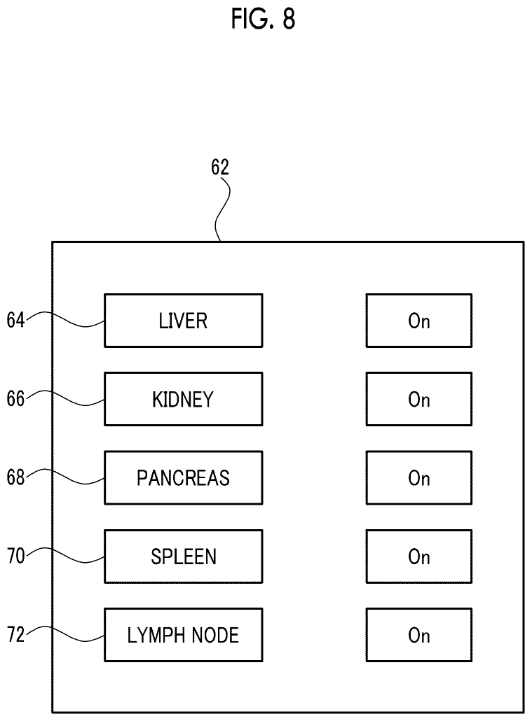

Hereinafter, Modification Example 3 will be described. In Modification Example 3, in a case in which the candidate having the score equal to or higher than the threshold value is not estimated, and a region in which the plurality of candidate images are displayed is designated, the display controller 22 displays the candidate list for the user to select the candidate on the display unit 18. Modification Example 3 will be described in detail with reference to FIGS. 7 and 8. FIG. 7 shows a display example of the candidate image group. FIG. 8 shows a selection screen displaying the candidate list for the scanning cross section.

As shown in FIG. 7, the screen 44 displays the ultrasound image 46 and the candidate image group 30. Here, the scores of the kidney cross section, the liver cross section, and the pancreas cross section are lower than the threshold value. Therefore, the screen 44 displays the candidate image group 30.

In this case, in a case in which the user operates the operation unit 26 to dispose a cursor 60 on the candidate image group 30 on the screen 44, the display controller 22 displays a selection screen 62 shown in FIG. 8 on the display unit 18. Disposing the cursor 60 on the candidate image group 30 is to designate the region in which the plurality of candidate images are displayed. For example, in a case in which the user moves the cursor 60 onto the candidate image group 30 on the screen 44, the display controller 22 displays the selection screen 62 on the display unit 18. The display controller 22 may display the selection screen 62 on the display unit 18 in a case in which the user touches or clicks the candidate image group 30. It should be noted that the display controller 22 may display the message 47 on the screen 44 as in the example shown in FIG. 5.

As shown in FIG. 8, the selection screen 62 displays scanning cross section names 64 to 72. Names of all candidates may be displayed on the selection screen 62, or the names of candidates selected through learning or the names of candidates based on a history may be displayed on the selection screen 62. The number of displayed names may be changed depending on the portion scanned with the ultrasound. Here, as an example, the liver, the kidney, the pancreas, the spleen, and the lymph node are displayed as the candidates for the scanning cross section.

In a case in which the user selects the scanning cross section name on the selection screen 62, the controller 28 confirms the scanning cross section having the selected scanning cross section name, as the cross section from which the ultrasound image 46 is obtained. For example, the ultrasound diagnostic apparatus 10 executes the examination specific to the scanning cross section having the selected scanning cross section name on the ultrasound image 46. The ultrasound diagnostic apparatus 10 may execute the examination even in a case in which the user does not issue the instruction to execute the examination. It goes without saying that the ultrasound diagnostic apparatus 10 may execute the examination in a case in which the user issues the instruction to execute the examination.

### Modification Example 4

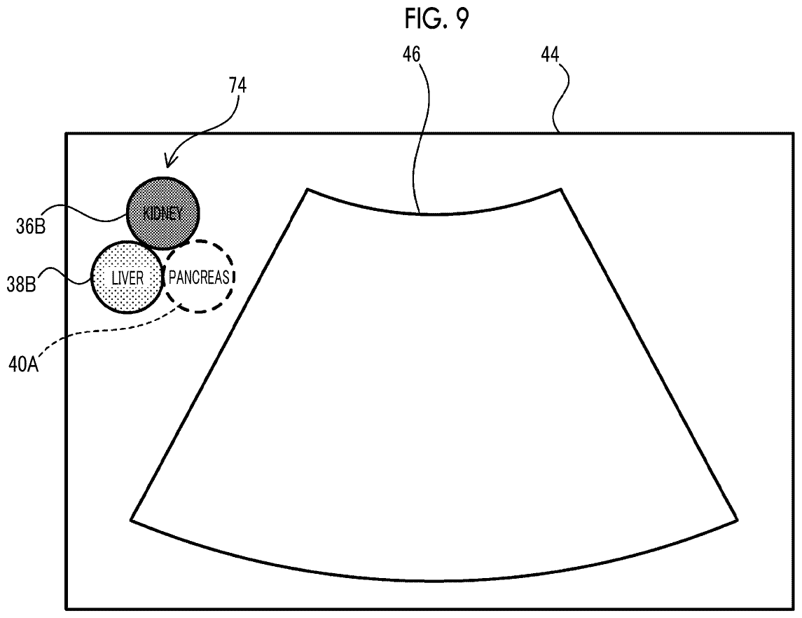

Hereinafter, Modification Example 4 will be described. In Modification Example 4, in a case in which the plurality of candidates are estimated by the recognition unit 20, the display controller 22 displays a candidate for the scanning cross section for which the examination is completed and a candidate for the scanning cross section for which the examination is not completed, on the display unit 18 in a distinguishable manner. Modification Example 4 will be described in detail with reference to FIG. 9. FIG. 9 shows a display example of the candidate image group.

The screen 44 displays the ultrasound image 46 and a candidate image group 74. The candidate image group 74 includes the candidate images 36B, 38B, and 40A as in the candidate image group 32 shown in FIG. 2. The kidney cross section is the first candidate, the liver cross section is the second candidate, and the pancreas cross section is the third candidate. Therefore, the candidate image 36B is disposed at the first candidate position, the candidate image 38B is disposed at the second candidate position, and the candidate image 40A is disposed at the third candidate position.

For example, it is assumed that the examination for the kidney is completed and the examinations for the liver and the pancreas are not completed at a point in time at which the candidate image group 74 is displayed on the screen 44. In this case, the display controller 22 displays the candidate image 36B and the candidate images 38B and 40A in a distinguishable manner. For example, the display controller 22 displays the candidate image corresponding to the part for which the examination is completed in gray-out. The display controller 22 displays the candidate image corresponding to the part for which the examination is not completed in an active state (non-gray-out state). In the example shown in FIG. 9, since the examination for the kidney is completed, the display controller 22 displays the candidate image 36B in gray-out. Since the examinations for the liver and the pancreas are not completed, the display controller 22 displays the candidate images 38B and 40A in an active state.

Even in a case in which the user selects the candidate image 36B displayed in gray-out, the controller 28 does not receive the selection. That is, even in a case in which the candidate image 36B is displayed at the first candidate position, the controller 28 does not receive the user's selection of the candidate image 36B. Therefore, the examination specific to the kidney is not executed.

For example, the controller 28 determines whether or not the examination for the kidney is completed by using a protocol assistant function or the like. For example, the workflow (that is, the examination protocol) is determined for each diagnostic target part. In the workflow, a type and an order of the examinations are predetermined, and the completion status of each examination is managed. The management of the workflow (for example, the management of the completion status of each examination) may be executed by the ultrasound diagnostic apparatus 10, or may be executed by the external apparatus (for example, a server) other than the ultrasound diagnostic apparatus 10. The controller 28 can determine whether or not the series of examinations on the kidney is completed, by referring to the workflow.

The display controller 22 may display a button (for example, a “Clear” button) that enables the selection of the candidate image displayed in gray-out on the screen 44. In a case in which the user presses the “Clear” button, the candidate image displayed in gray-out can be selected. For example, the candidate image 36B can be selected. In a case in which the user selects the candidate image 36B, the controller 28 receives the selection as the valid selection, and the ultrasound diagnostic apparatus 10 executes the examination specific to the kidney. For example, it is considered that the “Clear” button is pressed in a case of executing the re-measurement or re-examination.

It should be noted that, in a case in which the user operates the rotary encoder 42 or the like to dispose the candidate image 38B at the first candidate position and to issue the instruction to execute the examination, the ultrasound diagnostic apparatus 10 executes the examination specific to the liver. The same applies to the candidate image 40A.

### Modification Example 5

Hereinafter, Modification Example 5 will be described. In Modification Example 5, after the recognition unit 20 estimates the plurality of candidates and the user issues an instruction to execute re-recognition, the recognition unit 20 executes the recognition processing again on the ultrasound image. In this case, the recognition unit 20 estimates a new plurality of candidates for the scanning cross section, excluding the candidate having the highest score among the plurality of candidates. The excluded candidate (that is, the candidate having the highest score) is not included in the new plurality of candidates.

The recognition unit 20 may estimate the new candidates for the scanning cross section by executing the recognition processing again on the ultrasound image, excluding all candidates having the scores equal to or higher than the threshold value among the plurality of candidates. In this case, all the excluded candidates (that is, all the candidates having the scores equal to or higher than the threshold value) are not included in the new plurality of candidates.

The recognition unit 20 may estimate the new candidates for the scanning cross section by executing the recognition processing again on the ultrasound image, excluding a candidate shown by the candidate image disposed at the first candidate position. In this case, the excluded candidate (that is, the candidate shown by the candidate image disposed at the first candidate position) is not included in the new plurality of candidates.

In a case in which the new plurality of candidates are recognized by the recognition unit 20, the display controller 22 displays the candidate images showing the new plurality of candidates on the display unit 18.

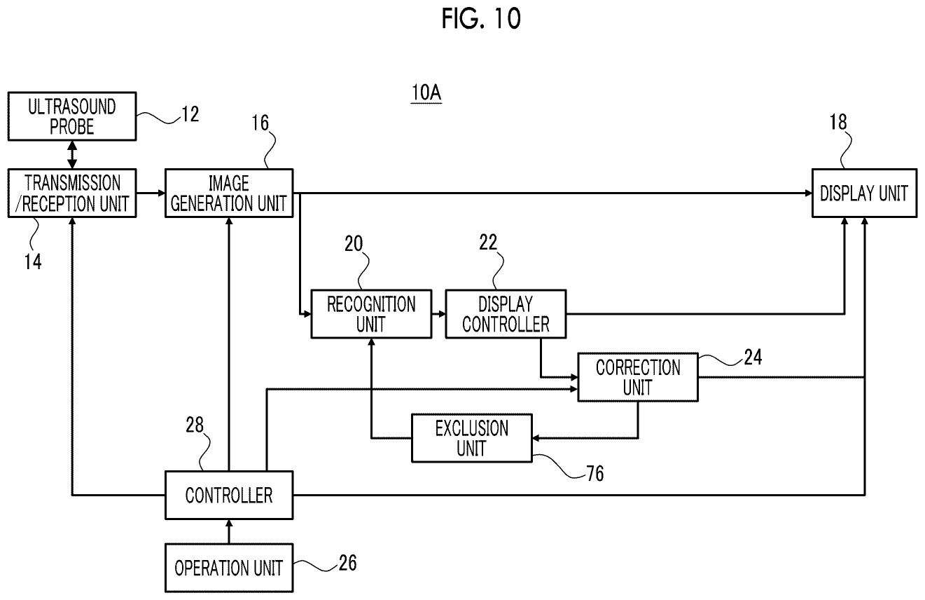

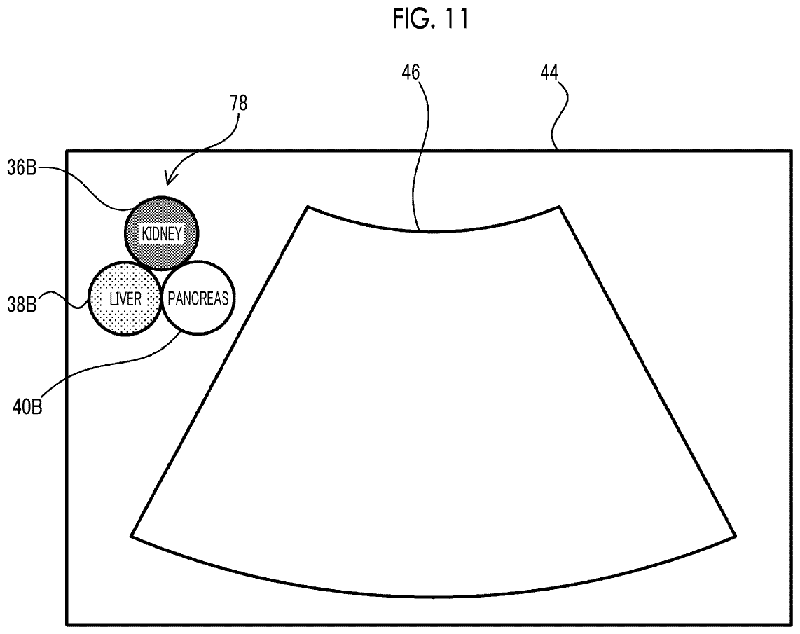

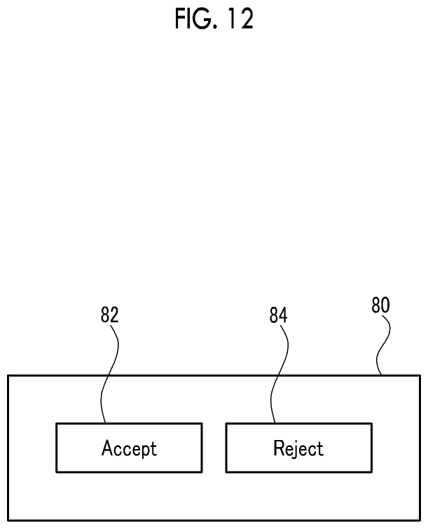

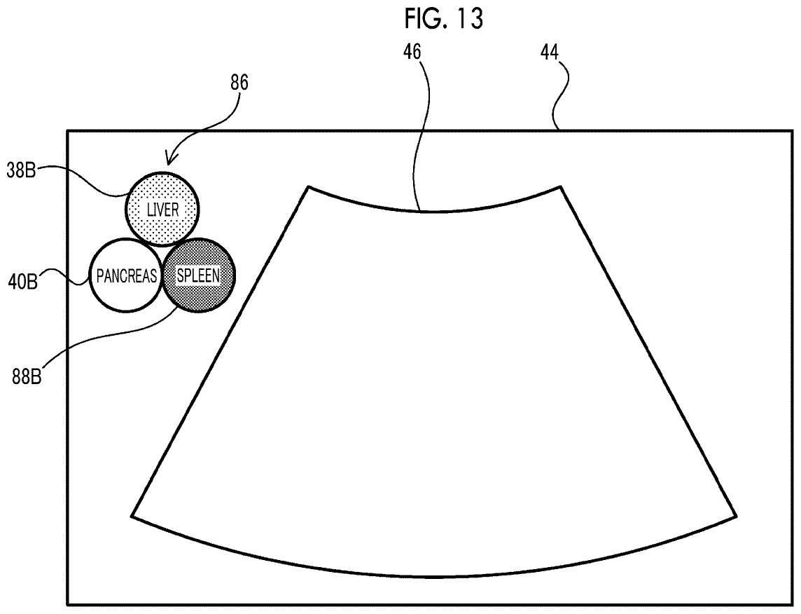

Modification Example 5 will be described in detail with reference to FIGS. 10 to 13. FIG. 10 is a block diagram showing a configuration of an ultrasound diagnostic apparatus 10A according to Modification Example 5. FIG. 11 shows a display example of the candidate image group. FIG. 12 shows a determination screen for the user to determine the candidate. FIG. 13 shows a display example of the candidate image group.

As shown in FIG. 10, the ultrasound diagnostic apparatus 10A includes an exclusion unit 76, in addition to the configuration of the ultrasound diagnostic apparatus 10. The configurations other than the exclusion unit 76 are the same as the configurations of the ultrasound diagnostic apparatus 10 shown in FIG. 1.

The exclusion unit 76 excludes the candidate having the highest score from the plurality of candidates estimated by the recognition unit 20, and outputs information indicating the excluded candidate to the recognition unit 20. As another example, the exclusion unit 76 may exclude all the candidates having the scores equal to or higher than the threshold value from the plurality of candidates estimated by the recognition unit 20, and output information indicating all the excluded candidates to the recognition unit 20. Further, as still another example, the exclusion unit 76 may exclude the candidate shown by the candidate image disposed at the first candidate position from the plurality of candidates estimated by the recognition unit 20, and output information indicating the excluded candidate to the recognition unit 20. The recognition unit 20 estimates the new candidates for the scanning cross section, excluding the candidates excluded by the exclusion unit 76.

FIG. 11 shows a result of a first estimation via the recognition unit 20 as an example. As shown in FIG. 11, the screen 44 displays the ultrasound image 46 and a candidate image group 78. The candidate image group 78 is an image group showing the results of the recognition on the ultrasound image 46, and includes candidate images 36B, 38B, and 40B. The candidate image 36B is an image showing the kidney cross section that is the candidate. The candidate image 38B is an image showing the liver cross section that is the candidate. The candidate image 40B is an image showing the pancreas cross section that is the candidate. Here, the frames of the candidate images 36B, 38B, and 40B are represented by a solid line. That is, the scores of the kidney cross section, the liver cross section, and the pancreas cross section are equal to or higher than the threshold value. The fact that the score is equal to or higher than the threshold value means that the reliability degree of the estimation of each candidate is high.

The display controller 22 displays a determination screen 80 shown in FIG. 12 on the display unit 18 after the candidate image group 78 is displayed on the screen 44. In a case in which the user issues an instruction to display the determination screen 80 by operating the operation unit 26, the display controller 22 may display the determination screen 80 on the display unit 18. In a case in which the user designates the candidate image group 78 (for example, in a case in which the user clicks or touches the candidate image group 78), the display controller 22 may display the determination screen 80 on the display unit 18.

As shown in FIG. 12, the determination screen 80 displays an “Accept” button 82 and a “Reject” button 84.

The result of the first estimation is confirmed in a case in which the user presses the “Accept” button 82. That is, a candidate group shown by the candidate image group 78 is confirmed. For example, the ultrasound diagnostic apparatus 10 executes the examination specific to the part corresponding to the candidate image disposed at the first candidate position on the ultrasound image 46.

In a case in which the user presses the “Reject” button 84, the instruction to execute the re-recognition is given to the recognition unit 20. The recognition unit 20 executes the recognition processing again on the ultrasound image 46 in response to the instruction. In this case, the exclusion unit 76 excludes the candidate having the highest score. The recognition unit 20 estimates the new plurality of candidates, excluding the candidate having the highest score. In the example shown in FIG. 11, the score of the kidney cross section is the highest. Therefore, the recognition unit 20 estimates the new plurality of candidates, excluding the kidney cross section. In this case, the kidney cross section is not included in the new plurality of candidates.

As another example, the recognition unit 20 may estimate the new plurality of candidates, excluding the candidates having the scores equal to or higher than the threshold value. In the example shown in FIG. 11, the scores of the kidney cross section, the liver cross section, and the pancreas cross section are equal to or higher than the threshold value. Therefore, the recognition unit 20 estimates the new plurality of candidates, excluding the kidney cross section, the liver cross section, and the pancreas cross section. In this case, the kidney cross section, the liver cross section, and the pancreas cross section are not included in the new plurality of candidates.

Further, as still another example, the recognition unit 20 may estimate the new plurality of candidates, excluding the candidate shown by the candidate image disposed at the first candidate position in the candidate image group 78. In the example shown in FIG. 11, the candidate image 36B showing the kidney cross section is disposed at the first candidate position. Therefore, the recognition unit 20 estimates the new plurality of candidates, excluding the kidney cross section. The user may change the arrangement order of the candidate images 36B, 38B, and 40B included in the candidate image group 78, to dispose the candidate images other than the candidate image 36B at the first candidate position. For example, in a case in which the candidate image 38B is disposed at the first candidate position, the recognition unit 20 estimates the new plurality of candidates, excluding the liver cross section. In this case, the liver cross section is not included in the new plurality of candidates.

FIG. 13 shows a result of a second estimation via the recognition unit 20. As shown in FIG. 13, the screen 44 displays the ultrasound image 46 and the candidate image group 86. The candidate image group 86 is an image group showing the results of the re-recognition of the ultrasound image 46, and is a candidate image group showing the new plurality of candidates.

Here, the new plurality of candidates are estimated excluding the candidate having the highest score among the plurality of candidates estimated by first recognition processing. That is, the new plurality of candidates are estimated excluding the kidney cross section. As a result of second recognition processing, the liver cross section, the pancreas cross section, and the spleen cross section are estimated as the candidates.

The candidate image group 86 includes candidate images 38B, 40B, and 88B. The candidate image 38B is an image showing the liver cross section that is the candidate. The candidate image 40B is an image showing the pancreas cross section that is the candidate. The candidate image 88B is an image showing the spleen cross section that is the candidate. Here, the frames of the candidate images 38B, 40B, and 88B are represented by a solid line. That is, the scores of the liver cross section, the pancreas cross section, and the spleen cross section are equal to or higher than the threshold value. The fact that the score is equal to or higher than the threshold value means that the reliability degree of the estimation of each candidate is high.

The display controller 22 displays a determination screen 80 shown in FIG. 12 on the display unit 18 after the candidate image group 86 is displayed on the screen 44. In a case in which the user issues the instruction to display the determination screen 80 by operating the operation unit 26, the display controller 22 may display the determination screen 80 on the display unit 18. In a case in which the user designates the candidate image group 86 (for example, in a case in which the user clicks or touches the candidate image group 86), the display controller 22 may display the determination screen 80 on the display unit 18.

The second estimation result is confirmed in a case in which the user presses the “Accept” button 82. That is, a candidate group shown by the candidate image group 86 is confirmed. For example, the ultrasound diagnostic apparatus 10 executes the examination specific to the part corresponding to the candidate image disposed at the first candidate position on the ultrasound image 46.

In a case in which the user presses the “Reject” button 84, the instruction to execute the re-recognition is given to the recognition unit 20. The recognition unit 20 executes the recognition processing again on the ultrasound image 46 in response to the instruction. In this case, the recognition unit 20 estimates the new plurality of candidates, excluding the candidates excluded by the exclusion unit 76. The same applies to the subsequent recognition processing.

### Modification Example 6

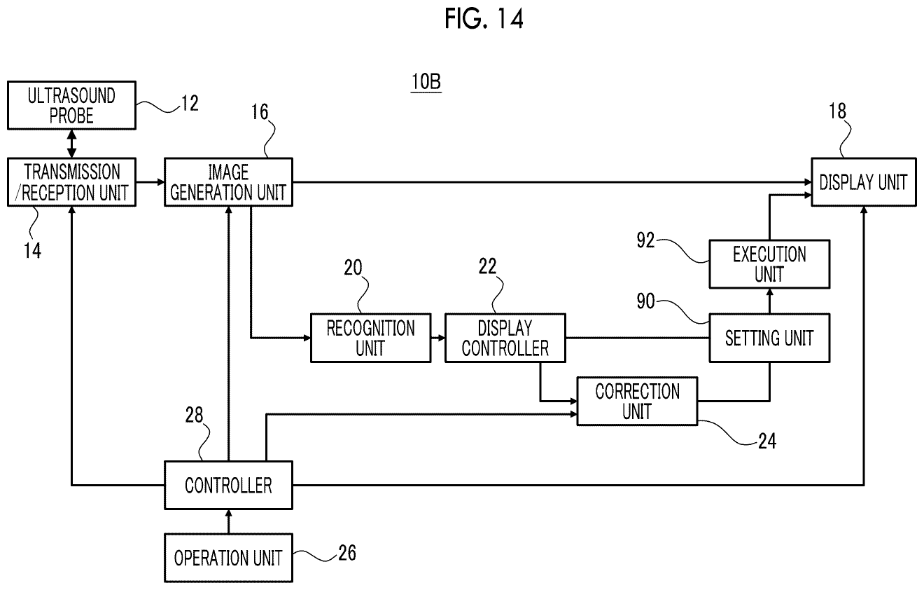

Modification Example 6 will be described with reference to FIG. 14. FIG. 14 is a block diagram showing a configuration of an ultrasound diagnostic apparatus 10B according to Modification Example 6.

The ultrasound diagnostic apparatus 10B includes a setting unit 90 and an execution unit 92, in addition to the configuration of the ultrasound diagnostic apparatus 10. The configurations other than the setting unit 90 and the execution unit 92 are the same as the configurations of the ultrasound diagnostic apparatus 10 shown in FIG. 1.

The setting unit 90 performs a setting related to the candidate for the scanning cross section. For example, the setting unit 90 performs the setting related to the first candidate among the plurality of candidates estimated by the recognition unit 20, on the ultrasound image. For example, the setting unit 90 sets a region of interest (ROI) that is a target of automatic measurement in the ultrasound image based on clinical knowledge information on the part corresponding to the candidate image disposed at the first candidate position. The clinical knowledge information is reference knowledge information related to the part, and is information determined in advance. For example, an ROI for designating a measurement region is determined in advance for each part. Specifically, in a case in which the part is the liver, depth information indicating a region in which a blood vessel is present corresponds to an example of the clinical knowledge information of the liver.

The execution unit 92 executes the examination in accordance with the setting made by the setting unit 90. For example, in a case in which the ROI is set in the ultrasound image, the execution unit 92 executes the examination such as measurement targeting for the ROI set in the ultrasound image.

For example, the ROI for the part corresponding to the candidate image disposed at the first candidate position is set in the ultrasound image, and the examination such as the measurement is executed targeting for the ROI. In this manner, the user can save the workload for manually setting the ROI related to the part having a high reliability degree of the estimation in the ultrasound image.

The setting unit 90 may perform the setting related to the candidate having the highest score on the ultrasound image, and the execution unit 92 may execute the examination targeting for the ultrasound image in accordance with the setting.

### Modification Example 7

In Modification Example 7, the processing according to the present embodiment may be applied to a body mark. For example, based on the result of the cross section recognition processing, a plurality of candidates for the body mark and the scores thereof may be calculated, and the rank of each candidate may be changed by the method according to the present embodiment.

The image generation unit 16, the recognition unit 20, the display controller 22, the correction unit 24, the controller 28, the exclusion unit 76, the setting unit 90, and the execution unit 92 can be realized by using hardware resources such as a processor and an electronic circuit, and devices such as a memory may be used as necessary in the realization. In addition, the image generation unit 16, the recognition unit 20, the display controller 22, the correction unit 24, the controller 28, the exclusion unit 76, the setting unit 90, and the execution unit 92 may be realized by, for example, a computer. That is, the image generation unit 16, the recognition unit 20, the display controller 22, the correction unit 24, the controller 28, the exclusion unit 76, the setting unit 90, and the execution unit 92 may be realized by the cooperation between the hardware resources such as a central processing unit (CPU) and a memory provided in the computer, and the software (program) that defines the operations of the CPU and the like. The program is stored in the storage device of the ultrasound diagnostic apparatus 10 or another storage device via a recording medium such as a CD or a DVD or through a communication path such as a network. As another example, the image generation unit 16, the recognition unit 20, the display controller 22, the correction unit 24, the controller 28, the exclusion unit 76, the setting unit 90, and the execution unit 92 may be realized by a digital signal processor (DSP), an application specific integrated circuit (ASIC), a field programmable gate array (FPGA), or the like. It goes without saying that, a graphics processing unit (GPU) or the like may be used. The image generation unit 16, the recognition unit 20, the display controller 22, the correction unit 24, the controller 28, the exclusion unit 76, the setting unit 90, and the execution unit 92 may be realized by a single apparatus or may be realized by a plurality of apparatuses.

It should be noted that the functions of the recognition unit 20, the display controller 22, the correction unit 24, the exclusion unit 76, the setting unit 90, and the execution unit 92 may be executed by an apparatus other than the ultrasound diagnostic apparatus 10 (for example, a personal computer or a server).

What is claimed is:

1. An ultrasound diagnostic apparatus comprising:
a recognition unit that executes recognition processing of a scanning cross section on an ultrasound image generated by being scanned with ultrasound, to estimate a plurality of candidates for a scanning cross section scanned with the ultrasound, and calculates a score indicating a certainty of the estimation for each candidate; and
a display controller that displays, on a display, candidate images showing a plurality of respective candidates having higher scores among the plurality of candidates in different display aspects for each score.

2. The ultrasound diagnostic apparatus according to claim 1,
wherein the display controller displays a plurality of the candidate images on the display in an arranged manner in an arrangement order corresponding to the scores, and changes the arrangement order of the plurality of candidate images in response to an instruction issued by a user to change the arrangement order to display the plurality of candidate images in an arranged manner on the display.

3. The ultrasound diagnostic apparatus according to claim 2, further comprising:
a reception unit that receives user's selection of a candidate having a score equal to or higher than a threshold value, and does not receive user's selection of a candidate having a score lower than the threshold value.

4. The ultrasound diagnostic apparatus according to claim 3,
wherein, in a case in which a candidate that corresponds to a candidate image displayed at a position corresponding to a specific rank in the arrangement order corresponding to the scores and that has the score equal to or higher than the threshold value is selected by the user, the reception unit receives the selection.

5. The ultrasound diagnostic apparatus according to claim 3,
wherein, in a case in which the candidate having the score lower than the threshold value is selected by the user, the reception unit receives the selection as valid selection depending on a content of processing executed after the selection.

6. The ultrasound diagnostic apparatus according to claim 2,
wherein, in a case in which a plurality of ultrasound images showing candidates having scores equal to or higher than a first threshold value are consecutively acquired, and the number of the consecutively acquired plurality of ultrasound images is equal to or higher than a second threshold value, the ultrasound diagnostic apparatus executes a next examination.

7. The ultrasound diagnostic apparatus according to claim 1,
wherein, in a case in which a candidate having a score equal to or higher than a threshold value is not estimated, the display controller displays information prompting a user to manually select the candidate on the display.

8. The ultrasound diagnostic apparatus according to claim 1,
wherein, in a case in which a candidate having a score equal to or higher than a threshold value is not estimated, and a region in which a plurality of the candidate images are displayed is designated, the display controller displays a candidate list for a user to select the candidate on the display.

9. The ultrasound diagnostic apparatus according to claim 1,
wherein the display controller displays a candidate for a cross section for which an examination is completed and a candidate for a cross section for which the examination is not completed, on the display in a distinguishable manner.

10. The ultrasound diagnostic apparatus according to claim 1,
wherein, in a case in which a user issues an instruction to execute re-recognition after the plurality of candidates are estimated by the recognition unit, the recognition unit executes the recognition processing on the ultrasound image again to estimate a new plurality of candidates for the scanning cross section, excluding a candidate having a highest score among the plurality of candidates, and
the display controller displays candidate images showing the new plurality of candidates on the display.

11. The ultrasound diagnostic apparatus according to claim 1, further comprising:
an execution unit that executes an examination in accordance with a setting related to a first candidate among the plurality of candidates.

12. Anon-transitory computer-readable storage medium storing a program causing a computer to function as:
a recognition unit that executes recognition processing of a scanning cross section on an ultrasound image generated by being scanned with ultrasound, to estimate a plurality of candidates for a scanning cross section scanned with the ultrasound, and calculates a score indicating a certainty of the estimation for each candidate; and
a display controller that displays, on a display, candidate images showing a plurality of respective candidates having higher scores among the plurality of candidates in different display aspects for each score.
